(19) United States
(12) Patent Application Publication
(10) Pub. No.: US 2024/0252585 A1
(43) Pub. Date: Aug. 1, 2024

(54) METHODS OF TREATING TRAUMATIC BRAIN INJURY AND RELATED NEUROLOGICAL CONDITIONS

(71) Applicant: The Board of Regents of the University of Oklahoma, Norman, OK (US)

(72) Inventors: Kelly M. Standifer, Norman, OK (US); Yong ZHANG, Edmond, OK (US)

(21) Appl. No.: 18/422,556

(22) Filed: Jan. 25, 2024

Related U.S. Application Data
(60) Provisional application No. 63/481,635, filed on Jan. 26, 2023.

(51) Int. Cl. A61K 38/10; A61K 31/444; A61K 31/451; A61K 31/454; A61K 31/4706; A61K 31/485; A61K 31/5025

(52) U.S. Cl. CPC A61K 38/10; A61K 31/444; A61K 31/451; A61K 31/454; A61K 31/4706; A61K 31/485; A61K 31/5025

(57) ABSTRACT

Methods of treating or mitigating, in a subject, traumatic brain injury (TBI), post-traumatic stress disorder (PTSD), and symptoms and conditions characteristic thereof, such as decreased cerebral blood flow (CBF), ischemia/hypoxia, and sensory, locomotor, generalized anxiety disorder (GAD), anxiety-like behaviors, and affective and cognitive dysfunction, by administering at least one Nociceptin Opioid Peptide (NOP) receptor partial agonist or antagonist to the subject. In at least certain non-limiting embodiments, subjects with TBI, PTSD, or both TBI and PTSD may be treated with at least one NOP receptor partial agonist or antagonist shortly after any type of TBI, thereby preventing or mitigating disrupted blood flow to the brain and the rapid and sustained impairments that would otherwise ensue.

### REFERENCE TO RELATED APPLICATIONS

The present application claims the benefit under 35 USC § 119(e) of U.S. Provisional Patent Application Ser. No. 63/481,635, filed Jan. 26, 2023. The entire contents of the above-referenced patent application(s) are hereby expressly incorporated herein by reference.

### STATEMENT REGARDING FEDERALLY SPONSORED RESEARCH OR DEVELOPMENT

Not Applicable.

### BACKGROUND

Traumatic brain injury (TBI) is a major cause of disability and death, responsible for ˜2.87 million emergency department visits, hospitalizations, and deaths in the United States in 2014. Because many mild TBI (mTBI) cases often go unreported, the number is likely up to 4 times higher. The nature, severity, and duration of symptoms experienced by TBI patients depend on the severity of the TBI: mild, moderate, or severe. TBI is the leading cause of death and disability in young adults (<40 years old) in the US. It is estimated by the Defense Medical Surveillance system that TBIs were sustained by over 439,609 U.S. service members around the world from 2000-2021; over 80% of them were mild. In fact, 80% of military TBIs occur to personnel that are not deployed, thus likely involving mechanisms of injury similar to those most experienced by civilians: falls, sports injuries, and motor vehicle accidents.

Mild TBIs are most common, and often occur in kids and young adults playing sports and in the elderly from falls; they often go unreported. If reported, they are checked for skull fractures and bleeding; if none are found, the patients are sent home to follow up if symptoms of headache, dizziness, imbalance, loss of memory, or inability to focus worsen. In ˜15% of patients with an mTBI, symptoms worsen and/or persist for months to years. Impaired motor and cognitive function, difficulties in maintaining balance and motor coordination, and anxiety are significant problems for those with TBI. Thus, many TBIs go undiagnosed or, upon confirmation of no structural injury or neurological deficits, patients are discharged with follow up as needed. However, for many patients, initial symptoms don't resolve and may worsen. TBI is often associated with cognitive deficits that may impede the patient's return to work for at least a year after the injury and up to five years later. Service members exposed to TBI are more prone to chronic conditions such as major depression, generalized anxiety disorder (GAD), and post-traumatic stress disorder (PTSD). Approximately 33% of military members with TBI also are diagnosed with PTSD. Research has indicated that levels of a natural substance in the brain increases after TBI and stays elevated for many days. This substance reduces blood flow in the brain, causing further damage to brain cells and their ability to ‘talk’ with each other properly.

PTSD and TBI fall on distinct ends of the spectrum of consequences to trauma exposure. TBI results from a physical trauma to the head or brain, while PTSD is a disordered physiological and psychological response to severe trauma that is not necessarily associated with physical injury. However, there is considerable overlap between symptoms that develop in TBI and PTSD patients. Both conditions commonly include insomnia, fatigue, irritability, pain, depression, anxiety, emotional numbing, avoidance, cognitive impairment, and hyperarousal. Both clinical and animal studies show that TBI is an obvious and important risk factor for the development of PTSD, and vice-versa. Preclinical studies examining comorbid PTSD and TBI are rare but have shown that comorbid TBI and PTSD produced some responses that were unique to the comorbidity and sensitized subjects initially exposed to one trauma, to subsequent effects of the other trauma.

Despite the obvious widespread need, the FDA has approved only two drugs to treat PTSD and none to treat TBI. The serious limits of effective therapeutics for PTSD and lack of therapeutics for TBI underscores the urgent need to find potential therapeutic targets for these conditions. Development of drugs that could be given after the injury that would prevent or reduce the severity of injury and injury-related problems is an important unmet need. It is to addressing this need that the present disclosure is directed.

### DETAILED DESCRIPTION

The present disclosure is directed to, in at least certain non-limiting embodiments, novel methods of treating TBI, PTSD, and symptoms and conditions characteristic thereof, such as decreased cerebral blood flow (CBF), ischemia/hypoxia, and sensory, locomotor, generalized anxiety disorder (GAD), anxiety-like behaviors, affective and cognitive dysfunction, by administering at least one NOP receptor partial agonist or antagonist to the subject. In at least certain non-limiting embodiments, patients with TBI, PTSD, or TBI+PTSD may be treated with at least one NOP receptor antagonist or partial agonist shortly after any type of TBI, thereby preventing or mitigating disrupted blood flow to the brain and the rapid and sustained impairments that follow.

Examples of NOP receptor partial agonists that can be used in the presently disclosed methods include, but are not limited to, PPL-138 (BU10038; 14b-(3-Phenylpropanoyl)-17-cyclo-propylmethyl-7,8-dihydronoroxymorphinone), AT-035 (Astraea Therapeutics), AT-001, AT-104, SER100 (ZP120), and others listed in U.S. Pat. No. 9,969,746 (including, for example, Compound Nos. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32, and the compounds included in claim 13 thereof), and in U.S. Pat. No. 10,112,924 (including, for example, Compound Nos. 1-414 of Tables 1, 2, and 3, and more particularly the compounds of claims 11-14 thereof), and the like, as well as any combinations thereof.

Examples of NOP receptor antagonists that can be used in the presently disclosed methods include, but are not limited to, LY2940094, MK-5757, JTC-801, SB612111, and any combinations thereof.
The following abbreviations may be used herein:
CBF: cerebral blood flow,
CCI: controlled cortical impact,
CSF: cerebrospinal fluid,
GAD: generalized anxiety disorder,
HPA: hypothalamic-pituitary-adrenal axis,
KO: knock-out,
mTBI: mild traumatic brain injury,
NOP: Nociceptin Opioid Peptide; Nociceptin/Orphanin FQ peptide,
PTSD: post-traumatic stress disorder, and
TBI: traumatic brain injury.

In previous work that investigated the temporal dynamics of pain and potential pain biomarkers in a preclinical model of PTSD, it was discovered that increased levels of the 17-amino acid neuropeptide Nociceptin/Orphanin FQ (N/OFQ) mediates allodynia and hyperalgesia in PTSD. N/OFQ acts as a ligand to the N/OFQ peptide (NOP) receptor (a.k.a., Nociceptin Opioid Peptide Receptor), which is present at peripheral nerve endings, mononuclear cells in the immune system, and neurons, astrocytes, and glial cells in the brain and spinal cord. NOP receptor/N/OFQ expression in the brain is particularly noteworthy; the N/OFQ-NOP receptor system modulates important functions such as pain sensitivity, hypothalamic-pituitary-adrenal axis (HPA) stress responses, cytokine production and release, neurovascular responsiveness, emotional processing, cognition, and vestibulomotor function. Many of these functions are altered in individuals suffering from PTSD and/or TBI. PTSD-induced allodynia and hyperalgesia could be reversed with an NOP receptor antagonist, and male NOP receptor knockout (KO) rats subjected to the PTSD protocol never developed allodynia at all. However, the absence of the NOP receptor failed to protect female rats from PTSD-induced allodynia, hyperalgesia, and anxiety-like behaviors. Clinical studies also found an association between the NOP receptor and PTSD. N/OFQ levels in CSF, brain, and plasma are up-regulated within 1-24 hr following percussive, stab, and blast-induced brain injuries. NOP receptor levels also are increased following blast TBI. N/OFQ and NOP receptor levels are elevated in the injured (but not uninjured) brain hemisphere 8 days after controlled cortical impact (CCI) brain that correlated with injury severity.

The normal uninjured brain can withstand short-term hypoxia; however, cerebral vasculature autoregulation is impaired in the injured brain. TBI-induced cerebral vasospasms reduced cerebral blood flow, apnea, hypoventilation, micro-hemorrhage, and reduced oxidative metabolism can lead to tissue ischemia, even when cerebral hypoperfusion and intracranial hypertension are absent. A recent TBI review paper noted that while altered cerebral blood flow (CBF) is one of the most common outcomes of TBI, “therapeutic procedures to enhance blood flow acutely are needed and could have significant clinical benefits for TBI patients.” As noted, the present disclosure addresses this critical need.

The treatments of the present disclosure are based on the observation that TBI causes acute elevation of N/OFQ neuropeptide, resulting in changes in localized NOP receptor signaling which causes reduced cerebral blood flow and ischemia, as well as the subsequent onset of many characteristic symptoms of TBI and PTSD. Continued elevation of N/OFQ neuropeptide levels maintains vestibular and sensory dysfunction and produces cognitive impairment.

Each patent, published patent application, and non-patent publication referenced in any portion of this application is expressly incorporated herein by reference in its entirety to the same extent as if the individual patent, published patent application, or non-patent publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities, and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. A range of 1 to 20 includes, for example, the numerals 1, 2, 3, 4, 5, 6, 7, 8, 9, 10, 11, 12, 13, 14, 15, 16, 17, 18, 19, and 20, and fractions between each integer, such as indicated above. A range of 6 to 24 includes, for example, the numerals 6, 7, 8, 9, 10, 11, 12, 13, 14, 15, 16, 17, 18, 19, 20, 21, 22, 23, 24, 25, and 26, and fractions between each integer, such as indicated above.

As noted above, any numerical range listed or described herein is intended to include, implicitly or explicitly, any number or sub-range within the range, particularly all integers, including the end points, and is to be considered as having been so stated. For example, “a range from 1.0 to 10.0” is to be read as indicating each possible number, including integers and fractions, along the continuum between and including 1.0 and 10.0, that is, having a minimum value equal to or greater than 1.0 and a maximum value equal to or less than 10.0, such as, for example, 3.25 to 8.65. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. Thus, even if a particular data point within the range is not explicitly identified or specifically referred to, it is to be understood that any data points within the range are to be considered to have been specified, and that the inventor(s) possessed knowledge of the entire range and the points within the range.

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, observer error, and combinations thereof, for example. The term “about” or “approximately,” where used herein when referring to a measurable value such as an amount, a temporal duration, and the like, is meant to encompass, for example, variations of ±20%, or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, at least 90% of the time, at least 91% of the time, at least 92% of the time, at least 93% of the time, at least 94% of the time, at least 95% of the time, at least 96% of the time, at least 97% of the time, at least 98% of the time, or at least 99% of the time.

As used herein any reference to “one embodiment” or “an embodiment” means that a particular element, feature, composition, structure, or characteristic described in connection with the embodiment is included in at least one embodiment. The appearances of the phrase “in one embodiment” in various places in the specification are not necessarily all referring to the same embodiment. Further, all references to one or more embodiments or examples are to be construed as non-limiting to the claims.

Where used herein, the pronoun “we” is intended to refer to all persons involved in a particular aspect of the work disclosed herein and as such may include non-inventor laboratory assistants and collaborators working under the supervision of the inventor.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the active agent) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “subject” and “patient” are used interchangeably herein and will be understood to refer to a warm-blooded animal, particularly a mammal. Non-limiting examples of animals within the scope and meaning of this term include dogs, cats, rabbits, rats, mice, guinea pigs, chinchillas, hamsters, ferrets, horses, pigs, goats, cattle, sheep, zoo animals, camels, llamas, non-human primates, including Old and New World monkeys and non-human primates (e.g., cynomolgus macaques, chimpanzees, rhesus monkeys, orangutans, and baboons), and humans.

The term “active agent” where used herein is intended to refer to a substance which possesses a biological activity relevant to the present disclosure, and particularly refers to therapeutic and diagnostic substances which may be used in methods described in the present disclosure. By “biologically active” is meant the ability to modify the physiological system of a cell, tissue, or organism without reference to how the active agent has its physiological effects. Where used herein, unless otherwise noted, the term “active agent” includes pharmaceutically-acceptable salts, hydrates, solvates, and amorphous solids thereof.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention. Non-limiting examples of modes of administration include inhalation, oral, topical, retrobulbar, subconjunctival, transdermal, parenteral, subcutaneous, intranasal, intramuscular, intraperitoneal, intravitreal, and intravenous routes, including both local and systemic applications. In addition, the compositions of the present disclosure may be designed to provide delayed, controlled, extended, and/or sustained release using formulation techniques which are well known in the art.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

Where used herein the term “drug combination” refers to a combination of drugs (compounds) which can be conjointly administered.

As used herein the terms “conjoint,” “conjointly,” “conjointly administered,” or “conjoint administration” refers to any form of administration of a combination of two or more different therapeutic compounds (also referred to herein as drugs or active agents) such that the second compound is administered while the previously administered therapeutic compound is still effective in the body, whereby the two or more compounds are simultaneously active in the patient. For example, the different therapeutic compounds can be administered either together in the same formulation (i.e., as a physical mixture), or in separate formulations, either concomitantly (at the same time) or sequentially. When administered sequentially the different compounds may be administered immediately in succession or separated by a suitable duration of time.

The term “coadministration” refers to administration of two or more active agents, e.g., an NOP receptor-targeted composition as described herein and another active agent. The timing of coadministration depends in part on the combination and compositions administered and can include administration at the same time, just prior to, or just after the administration of one or more additional therapies. “Coadministration” is meant to include simultaneous or sequential administration of the compound and/or composition individually or in combination. Thus, the preparations can also be combined, when desired, with other active substances (e.g., to reduce metabolic degradation). For example, the compositions described herein can be used in combination with one another, or with other active agents known to be useful in treating TBI, and co-occurring conditions thereof, e.g., PTSD.

The term “effective amount” refers to an amount of an active agent which is sufficient to exhibit a detectable therapeutic effect without excessive adverse side effects (such as toxicity, irritation and allergic response) commensurate with a reasonable benefit/risk ratio when used in the manner disclosed herein. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition, disease or symptom thereof. A detectable or measurable improvement includes a subjective or objective decrease, reduction, inhibition, suppression, limit or control in the occurrence, frequency, severity, progression, or duration of the condition or disease, or an improvement in a symptom or an underlying cause or a consequence of the disease, or a reversal of the disease. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most or all adverse symptoms, complications, consequences or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

The compounds of the present disclosure may be combined with one or more pharmaceutically-acceptable excipients, including carriers, vehicles, diluents, and adjuvants which may improve solubility, deliverability, dispersion, stability, and/or conformational integrity of the compounds or conjugates thereof.

“Pharmaceutically acceptable salts” means salts of active agent compounds disclosed herein which are pharmaceutically acceptable, as defined above, and which possess the desired pharmacological activity. Such salts include (but are not limited to) acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include (but are not limited to) base addition salts which may be formed when acidic protons present are capable of reacting with inorganic or organic bases. Acceptable inorganic bases include (but are not limited to) sodium hydroxide, sodium carbonate, potassium hydroxide, aluminum hydroxide and calcium hydroxide. Acceptable organic bases include (but are not limited to) ethanolamine, diethanolamine, triethanolamine, tromethamine, N-methylglucamine and the like. It should be recognized that the particular anion or cation forming a part of any salt of the present disclosure is not critical, so long as the salt, as a whole, is pharmacologically acceptable. Additional non-limiting examples of pharmaceutically acceptable salts and their methods of preparation and use are shown in Handbook of Pharmaceutical Salts: Properties, and Use (P. H. Stahl & C. G. Wermuth eds., Verlag Helvetica Chimica Acta, 2002).

The active agents of the present disclosure may be present in the pharmaceutical compositions (alone or in combination) at any concentration that allows the pharmaceutical composition to function in accordance with the present disclosure; for example, but not by way of limitation, the compound(s) may be present in a carrier, diluent, or buffer solution in a wt/wt or vol/vol range having a lower level selected from about 0.00001%, about 0.0001%, about 0.005%, about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, and about 2.0%; and an upper level selected from about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90%, and about 95%. Non-limiting examples of particular wt/wt or vol/vol ranges include a range of from about 0.0001% to about 95%, a range of from about 0.001% to about 75%; a range of from about 0.005% to about 50%; a range of from about 0.01% to about 40%; a range of from about 0.05% to about 35%; a range of from about 0.1% to about 30%; a range of from about 0.1% to about 25%; a range of from about 0.1% to about 20%; a range of from about 1% to about 15%; a range of from about 2% to about 12%; a range of from about 5% to about 10%; and the like. Any other range that includes a lower level selected from the above-listed lower level concentrations and an upper level selected from the above-listed upper level concentrations also falls within the scope of the present disclosure. Percentages used herein may be weight percentages (wt %) or volume percentages (vol %).

In certain non-limiting embodiments, an effective amount or therapeutic dosage of a pharmaceutical composition of the present disclosure contains sufficient active agent to deliver from about 0.001 μg/kg to about 100 mg/kg (weight of active agent/body weight of the subject). For example, the composition will deliver about 0.01 μg/kg to about 50 mg/kg, and more particularly about 0.1 μg/kg to about 10 mg/kg, and more particularly about 1 μg/kg to about 1 mg/kg of active agent. Practice of a method of the present disclosure may comprise administering to a subject an effective amount of the active agent in any suitable systemic and/or local formulation, in an amount effective to deliver the therapeutic dosage of the active agent. In certain non-limiting embodiments, an effective dosage may be, in a range of about 1 μg/kg to about 1 mg/kg of the active agent.

Certain non-limiting embodiments of the present disclosure are directed to a method that comprises administering to a subject in need thereof any of the pharmaceutical compositions disclosed or otherwise contemplated herein.

The pharmaceutical compositions may be administered via any mechanisms disclosed herein or otherwise contemplatable by a person having ordinary skill in the art. In one non-limiting embodiment, the administration occurs via an inhaler, which aerosolizes the active agents.

The pharmaceutical compositions of the present disclosure may be administered for any purpose disclosed or otherwise contemplated herein, as well as for any purpose within the purview of a person having ordinary skill in the art. In one non-limiting embodiment, the pharmaceutical compositions are administered in a method of treating or reducing the occurrence of cancer. However, this treatment method is not to be construed as limiting of the present disclosure, and any diseases, disorders, or conditions disclosed herein or otherwise contemplatable by a person having ordinary skill in the art (given the subject application) which may derive a therapeutic effect by treatment with the compositions disclosed herein also fall within the scope of the methods of the present disclosure.

Practice of the methods of the present disclosure may comprise administering to a subject therapeutically effective amounts of the active agents in any suitable systemic and/or local formulation, in an amount effective to deliver the dosages listed herein. The dosage can be administered, for example but not by way of limitation, on a one-time basis, or administered at multiple times (for example but not by way of limitation, from one to five times per day, or once or twice per week), or continuously via a venous drip, depending on the desired therapeutic effect. In one non-limiting example of a therapeutic method of the present disclosure, the active agent is provided in an IV infusion in a range of from about 0.01 mg/kg to about 10 mg/kg of body weight once a day.

The compositions and dosage forms of the present disclosure can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day. The duration of treatment (i.e., the period of time over which the composition is administered) can vary depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. Where used herein, unless otherwise indicated, the dosage amount refers to the amount of active agent, or active pharmaceutical ingredient (API), that is administered to the subject.

The dosage of an administered active agent for humans will vary depending upon factors such as (but not limited to) the patient's age, weight, height, sex, general medical condition, and previous medical history. In certain non-limiting embodiments, where the active agent is administered by injection or infusion, the recipient may be provided with a dosage of the active agent that is in the range of from about 1 mg to about 1000 mg, and it may be administered as a single infusion or multiple injections, although a lower or higher dosage also may be administered. In certain non-limiting embodiments, the dosage may be in the range of from about 25 mg to about 100 mg of the active agent per square meter (m) of body surface area for a typical adult, although a ² lower or higher dosage also may be administered. Non-limiting examples of dosages of the active agent that may be administered to a human subject include, but are not limited to, those in ranges of about 1 to about 1000 mg, about 1 to about 600 mg, about 1 to about 500 mg, about 1 to about 400 mg, about 1 to about 300 mg, about 1 to about 200 mg, about 100 to about 600 mg, about 100 to about 500 mg, about 100 to about 400 mg, about 100 to about 300 mg, about 100 to about 200 mg, about 150 to about 600 mg, about 150 to about 500 mg, about 150 to about 400 mg, about 150 to about 300 mg, about 150 to about 250 mg, about 150 to about 200 mg, about 200 to about 750 mg, about 200 to about 600 mg, about 200 to about 500 mg, about 200 to about 400 mg, about 200 to about 300 mg, and about 200 to about 250 mg, or any subrange within any of the aforementioned ranges. Dosages may be repeated as needed, for example (but not by way of limitation), once per week for 4-10 weeks, once per week for 8 weeks, or once per week for 4 weeks. It may also be given less frequently, such as (but not limited to) every other week for several months, or more frequently, such as twice weekly or by continuous infusion.

In certain non-limiting embodiments, the present disclosure is directed to a dosing regimen comprising multiple dosing cycles (e.g., wherein the first dosing cycle is a step-up, fractionated dosing cycle). In some non-limiting embodiments, the dose may range from about 50 mg to about 200 mg (e.g., from about 50 mg to about 175 mg, from about 50 mg to about 150 mg, from about 50 mg to about 125 mg, from about 50 mg to about 100 mg, from about 50 mg to about 75 mg, from about 50 mg to about 70 mg, from about 52 mg to about 100 mg, from about 52 mg to about 75 mg, from about 50 mg to about 180 mg, from about 55 mg to about 150 mg, from about 55 mg to about 100 mg, from about 55 mg to about 70 mg, from about 55 mg to about 65 mg, from about 58 mg to about 62 mg; e.g., about 60 mg, or any subrange within any of the aforementioned ranges). In some non-limiting embodiments, the dose may be about 60 mg. In some non-limiting embodiments, the dose is about 1 mg. In some non-limiting embodiments, the dose is about 2 mg.

In some non-limiting embodiments, the dose is from about 20 mg to about 200 mg (e.g., from about 20 mg to about 175 mg, from about 20 mg to about 150 mg, from about 20 mg to about 100 mg, from about 20 mg to about 75 mg, from about 30 mg to about 175 mg, from about 40 mg to about 175 mg, from about 45 mg to about 175 mg, from about 50 mg to about 175 mg, from about 30 mg to about 150 mg, from about 40 mg to about 100 mg, from about 45 mg to about 75 mg, from about 50 mg to about 70 mg, from about 55 mg to about 65 mg, from about 58 mg to about 62 mg; about 20 mg, about 30 mg, about 45 mg, or e.g., about 60 mg, or any subrange within any of the aforementioned ranges). In some non-limiting embodiments, the dose is from about 12 mg to about 48 mg (e.g., from about 12 mg to about 42 mg, from about 12 mg to about 36 mg, from about 12 mg to about 30 mg, from about 18 mg to about 48 mg, from about 18 mg to about 42 mg, from about 24 mg to about 42 mg, from about 27 mg to about 42 mg, from about 24 mg to about 36 mg, from about 27 mg to about 33 mg, from about 28 mg to about 32 mg; e.g., about 24 mg, about 27 mg, about 30 mg, about 33 mg, or about 36 mg, or any subrange within any of the aforementioned ranges).

In some non-limiting embodiments, the dosing regimen comprises administration of a dose in a range of from about 100 mg to about 750 mg (e.g., from about 100 mg to about 725 mg, from about 100 mg to about 700 mg, from about 100 mg to about 675 mg, from about 100 mg to about 650 mg, from about 100 mg to about 625 mg, from about 100 mg to about 600 mg, from about 100 mg to about 575 mg, from about 100 mg to about 550 mg, from about 100 mg to about 525 mg, from about 100 mg to about 500 mg, from about 100 mg to about 475 mg, from about 100 mg to about 450 mg, from about 100 mg to about 425 mg, from about 100 mg to about 400 mg, from about 100 mg to about 375 mg, from about 100 mg to about 350 mg, from about 100 mg to about 325 mg, from about 100 mg to about 300 mg, from about 100 mg to about 275 mg, from about 100 mg to about 250 mg, or from about 100 mg to about 225 mg, from about 100 mg to about 200 mg, from about 100 mg to about 175 mg, from about 100 mg to about 150 mg, or from about 100 mg to about 125 mg, or any subrange within any of the aforementioned ranges).

In some non-limiting embodiments, the dosing regimen comprises administration of a dose in a range of from about 200 mg to about 750 mg (e.g., from about 200 mg to about 725 mg, from about 200 mg to about 700 mg, from about 200 mg to about 675 mg, from about 200 mg to about 650 mg, from about 200 mg to about 625 mg, from about 200 mg to about 600 mg, from about 200 mg to about 575 mg, from about 200 mg to about 550 mg, from about 200 mg to about 525 mg, from about 200 mg to about 500 mg, from about 200 mg to about 475 mg, from about 200 mg to about 450 mg, from about 200 mg to about 425 mg, from about 200 mg to about 400 mg, from about 200 mg to about 375 mg, from about 200 mg to about 350 mg, from about 200 mg to about 325 mg, from about 200 mg to about 300 mg, from about 200 mg to about 275 mg, from about 200 mg to about 250 mg, or from about 200 mg to about 225 mg, or any subrange within any of the aforementioned ranges).

In some non-limiting embodiments, the dosing regimen comprises administration of a dose in a range of from about 300 mg to about 750 mg (e.g., from about 300 mg to about 725 mg, from about 300 mg to about 700 mg, from about 300 mg to about 675 mg, from about 300 mg to about 650 mg, from about 300 mg to about 625 mg, from about 300 mg to 600 mg, from about 300 mg to about 575 mg, from about 300 mg to about 550 mg, from about 300 mg to about 525 mg, from about 300 mg to about 500 mg, from about 300 mg to about 475 mg, from about 300 mg to about 450 mg, from about 300 mg to about 425 mg, from about 300 mg to about 400 mg, from about 300 mg to about 375 mg, from about 300 mg to about 350 mg, or from about 300 mg to about 325 mg, or any subrange within any of the aforementioned ranges).

In some non-limiting embodiments, the dosing regimen comprises administration of a dose in a range of from about 400 mg to about 750 mg (e.g., from about 400 mg to about 725 mg, from about 400 mg to about 700 mg, from about 400 mg to about 675 mg, from about 400 mg to about 650 mg, from about 400 mg to about 625 mg, from about 400 mg to about 600 mg, from about 400 mg to about 575 mg, from about 400 mg to about 550 mg, from about 400 mg to about 525 mg, from about 400 mg to about 500 mg, from about 400 mg to about 475 mg, from about 400 mg to about 450 mg, or from about 400 mg to about 425 mg, or any subrange within any of the aforementioned ranges).

In some non-limiting embodiments, the dosing regimen comprises administration of a dose in a range of from about 500 mg to about 750 mg (e.g., from about 500 mg to about 725 mg, from about 500 mg to about 700 mg, from about 500 mg to about 675 mg, from about 500 mg to about 650 mg, from about 500 mg to about 625 mg, from about 500 mg to about 600 mg, from about 500 mg to about 575 mg, from about 500 mg to about 550 mg, or from about 500 mg to about 525 mg, or any subrange within any of the aforementioned ranges).

In some non-limiting embodiments, the dosing regimen comprises administration of a dose in a range of from about 600 mg to about 750 mg (e.g., from about 600 mg to about 725 mg, from about 600 mg to about 700 mg, from about 600 mg to about 675 mg, from about 600 mg to about 650 mg, from about 600 mg to about 625 mg, or any subrange within any of the aforementioned ranges).

In some non-limiting embodiments, the active agent is provided in a concentration of about 1 nM, about 5 nM, about 10 nM, about 25 nM, about 50 nM, about 75 nM, about 100 nM, about 150 nM, about 200 nM, about 250 nM, about 300 nM, about 350 nM, about 400 nM, about 500 nM, about 550 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 75 μM, about 80 μM, about 90 μM, about 100 μM, about 125 μM, about 150 μM, about 175 μM, about 200 μM, about 250 M, about 300 μM, about 350 μM, about 400 μM, about 500 μM, about 600 μM, about 700 μM, about 750 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 400 mM, about 500 mM, about 600 mM, about 700 mM, about 800 mM, about 900 mM, about 1000 mM, about 1 M, about 1.1 M, about 1.2 M, about 1.3 M, about 1.4 M, about 1.5 M, about 1.6 M, about 1.7 M, about 1.8 M, about 1.9 M, about 2 M, about 3 M, about 4 M, about 5 M, about 6 M, about 7 M, about 8 M, about 9 M, about 10 M, about 15 M, about 20 M, about 25 M, about 30 M, about 35 M, about 40 M, about 45 M, about 50 M, about 75 M, about 100 M, or any range in between any two of the aforementioned concentrations, including said two concentrations as endpoints of the range, or any number in between any two of the aforementioned concentrations.

When administered orally, the active agent composition may be protected from digestion. This can be accomplished either by complexing the active agent with a composition to render it resistant to acidic and enzymatic hydrolysis or by packaging the active agent in an appropriately resistant carrier such as (but not limited to) a liposome, e.g., such as shown in U.S. Pat. No. 5,391,377.

In certain non-limiting embodiments, the different therapeutic compounds of the disclosure can be administered within one hour of each other, within two hours of each other, within 3 hours of each other, within 6 hours of each other, within 12 hours of each other, within 24 hours of each other, within 36 hours of each other, within 48 hours of each other, within 72 hours of each other, or more. Thus, an individual who receives such treatment can benefit from a combined effect of the different therapeutic compounds.

The active agents of the present disclosure can be administered to a subject by any of a number of effective routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally, or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example, as a patch applied to the skin); and topically (for example, as a cream, ointment, or spray applied to the skin, or as an eye drop). The compounds may also be formulated for inhalation. In certain non-limiting embodiments, a compound may be simply dissolved or suspended in sterile water. Oral formulations may be formulated such that the active agents pass through a portion of the digestive system before being released; for example, the active agent may not be released until reaching the small intestine or the colon. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

Tablets and other solid dosage forms of the pharmaceutical compositions, such as dragees, capsules (including sprinkle capsules and gelatin capsules), pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions that can be dissolved in sterile water or some other sterile injectable medium immediately before use. These compositions may also optionally contain opacifying agents and may be of a composition that releases the active ingredient(s) only, or preferentially (but not by way of limitation), in a certain portion of the gastrointestinal tract, optionally, in a delayed manner. Non-limiting examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups, and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax, or a salicylate, and which is solid at room temperature, but liquid at body temperature and, therefore, will melt in the rectum or vaginal cavity and release the active compound.

Formulations which are suitable for vaginal administration also include pessaries, tampons, creams, gels, pastes, foams or spray formulations containing such carriers as are known in the art to be appropriate. In certain non-limiting embodiments, the active agents of the present disclosure can be formulated into suppositories, slow release formulations, or intrauterine delivery devices (IUDs).

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants that may be required. The ointments, pastes, creams, and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions, and the like are also contemplated as being within the scope of the present disclosure. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A particular (but non-limiting) route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, and intraspinal and intrasternal injection and infusion.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical transdermal administration, the agents are formulated into ointments, creams, salves, powders, and gels. Transdermal delivery systems can also include (for example but not by way of limitation) patches. The present compositions can also be administered in sustained delivery or sustained release mechanisms. For example, biodegradable microspheres or capsules or other biodegradable polymer configurations capable of sustained delivery can be included herein.

The compositions of the present disclosure may be formulated as implants, in the form of either biodegradable microparticles or small squared films comprising the microparticles of the present disclosure. The following describes methods of making such implants. Microparticles (e.g., 5-100 micrometers) containing different loadings of the compounds of the present disclosure may be prepared by spray drying suspensions of the nanocrystals of the compounds and a biodegradable polymer (for example, polylactic acid of molecular weights 50,000-100,000) or polylactic-co-glycolic acid copolymer (e.g., proportions 75:25 or 50:50). Microparticles may contain, e.g., 10-50% wt/wt drug: polymer and can be implanted alone, or in a biodegradable film, e.g., as an implantable chitosan-egg phosphatidylcholine (ePC) films. To make such chitosan-egg phosphatidylcholine (ePC) films, chitosan flakes and ePC can be dissolved in a 1% acetic acid at a ratio of 1:0.8 (wt/wt). Microparticles containing the drug in nanocrystal form are dispersed in the chitosan-ePC solution in different proportions (e.g., 1:3, 1:5, 1:7 and 1:10 wt/wt) to achieve the release of different drug doses. The resulting microparticle-chitosan-ePC suspension can be poured into a Teflon dish to have a 2-3 mm thickness and allowed to dry in a covered dessicator for 5 days. After the films are dry, they can be cut into small squares of 15×15 mm. The implants ² can be made in different forms, including but not limited to thin films, rods, and wafers. Other biodegradable polymers that can be used to make implants include, but are not limited to, poly-lactic acid, poly-lactic-co-glycolic acid copolymer, poly-caprolactone, poly-sebacic acid, poly-adipic acid, poly (3-hydroxybutyric acid), poly(3-hydroxybutyrate-co-3-hydroxyvalerate, poly-trimethylene carbonate, chitosan, chitin, gelatin, collagen, and hyaluronic acid.

In non-limiting embodiments, gels comprising the active agents of the present disclosure can be made by combining the active agents in various proportions to a sodium alginate gel base or carbomer jelly base to form a homogeneous gel suitable for topical application. In non-limiting embodiments, ointments comprising the active agents of the present disclosure can be made by combining the active agents in various proportions to a Hydrophilic Petrolatum USP base, Lanolin, USP base or to Polyethylene glycol ointment, NF to form a homogeneous ointment suitable for topical application.

In non-limiting embodiments, creams comprising the active agents of the present disclosure can be made by combining the active agents in various proportions in suspension in water and glycerin (e.g., 20:1 parts) and emulsified in a mixture of e.g., Lanolin, Beeswax USP-NF and Cetyl alcohol, plus a Tween 80 and Span 80.

Several gel, ointment, and/or cream compositions that can be used are shown in García-Contreras L, Abu-Izza K, Lu DR. “Biodegradable cisplatin microspheres for direct brain injection: Preparation and characterization.” Pharm Dev Technol (1997) 2(1): 53-65.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another non-limiting aspect, the device for delivering the formulation to respiratory tissue is an inhaler in which the formulation vaporizes. Other liquid delivery systems include (for example but not by way of limitation) air jet nebulizers.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another non-limiting aspect, the device for delivering the formulation to respiratory tissue is an inhaler in which the formulation vaporizes. Other liquid delivery systems include (for example, but not by way of limitation) air jet nebulizers.

Pharmaceutical compositions suitable for parenteral administration comprise one or more active compounds in combination with one or more pharmaceutically acceptable sterile isotonic aqueous or nonaqueous solutions, dispersions, suspensions, or emulsions, or sterile powders which may be reconstituted into sterile injectable solutions or dispersions just prior to use, which may contain antioxidants, buffers, bacteriostats, solutes which render the formulation isotonic with the blood of the intended recipient, or suspending or thickening agents.

Non-limiting examples of suitable aqueous and nonaqueous carriers that may be employed in the pharmaceutical compositions of the present disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In certain non-limiting embodiments, these compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Non-limiting examples of other biodegradable polymers include poly(orthoesters) and poly(anhydrides). Depot injectable formulations are also prepared by entrapping the drug in liposomes or microemulsions that are compatible with body tissue.

As noted, effective amounts of the active agents may be administered orally, in the form of a solid or liquid preparations such as capsules, pills, tablets, lozenges, melts, powders, suspensions, solutions, elixirs, or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition may contain a solid carrier, such as a gelatin or an adjuvant. The tablet, capsule, and powder may contain from about 0.05 to about 95% of the active substance compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition particularly contains from about 0.005 to about 95% by weight of the active agent(s). For example, a dose of about 10 mg to about 1000 mg once or twice a day could be administered orally.

In another non-limiting embodiment, the active agents of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the active agents in an aqueous or non-aqueous pharmaceutically acceptable solvent which may also contain suspending agents, sweetening agents, flavoring agents, and preservative agents as are known in the art.

In one non-limiting aspect, the active agent is incorporated in lipid monolayers or bilayers, such as (but not limited to) liposomes. Liposomes and liposomal formulations can be prepared according to standard methods and are also well known in the art, such as (but not limited to) those disclosed in U.S. Pat. Nos. 6,110,490; 6,096,716; 5,283,185; 5,279,833; 4,235,871; 4,501,728; and 4,837,028.

In one non-limiting aspect, the compositions are prepared with carriers that will protect the active agent against rapid elimination from the body, such as (but not limited to) a controlled release formulation, including implants and microencapsulated delivery systems. Biodegradable, biocompatible polymers can be used, such as (but not limited to) ethylene vinyl acetate, polyanhydrides, polyglycolic acid, collagen, polyorthoesters, and polylactic acid. Methods for preparation of such formulations will be apparent to those skilled in the art.

The active agents in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active agent is combined in a mixture with one or more pharmaceutically suitable excipients. Sterile phosphate-buffered saline is one non-limiting example of a pharmaceutically suitable excipient.

The acid addition salts may include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, cam phorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N?-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Non-limiting examples of routes of administration of the compositions described herein include parenteral injection, e.g., by subcutaneous, intramuscular, or transdermal delivery. Other forms of injection include (but are not limited to) intravenous, intraarterial, intralymphatic, intrathecal, intraocular, intranasal, intracranial, intracerebral, intraperitoneal, or intracavitary injection. In parenteral administration, the compositions will be formulated in a unit dosage injectable form such as (but not limited to) a solution, suspension, or emulsion, in association with a pharmaceutically acceptable excipient. Such excipients are inherently nontoxic and nontherapeutic. Non-limiting examples of such excipients include saline, Ringer's solution, dextrose solution, and Hanks' solution. Nonaqueous excipients such as (but not limited to) fixed oils and ethyl oleate may also be used. An alternative non-limiting excipient is 5% dextrose in saline. The excipient may contain minor amounts of additives such as (but not limited to) substances that enhance isotonicity and chemical stability, including buffers and preservatives. The active agents can be delivered or administered alone or as pharmaceutical compositions by any means known in the art, such as (but not limited to) systemically, regionally, or locally, for example by intraarterial, intrathecal (IT), intravenous (IV), parenteral, intrapleural cavity, topical, oral, or local administration, as subcutaneous, intratracheal (e.g., by aerosol), or transmucosal administration (e.g., buccal, bladder, vaginal, uterine, rectal, and/or nasal mucosa). Administration can also be localized directly into a tumor. Administration into the systemic circulation by intravenous, inhalation, mucosal, or subcutaneous administration is typical. Intravenous administration can be, for example (but not by way of limitation), by infusion over a period such as (but not limited to) 30-90 min or by a single bolus injection, or by other regimens as described elsewhere herein.

For parenteral administration, for example, the active agents may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When an effective amount of the active agents is administered by intravenous, cutaneous, or subcutaneous injection, the compound is particularly in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the active agent, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical compositions of the present disclosure may also contain stabilizers, preservatives, buffers, antioxidants, or other additives known to those of skill in the art.

As noted, particular amounts and modes of administration can be determined by one skilled in the art. One skilled in the art of preparing formulations can readily select the proper form and mode of administration, depending upon the particular characteristics of the active agents selected, the condition to be treated, the stage of the condition, and other relevant circumstances using formulation technology known in the art, described, for example, in Remington: The Science and Practice of Pharmacy, 22nd ed.

Additional pharmaceutical methods may be employed to control the duration of action of the active agents. Increased half-life and/or controlled release preparations may be achieved through the use of proteins or polymers to conjugate, complex with, and/or absorb the active agents as discussed previously herein. The controlled delivery and/or increased half-life may be achieved by selecting appropriate macromolecules (for example but not by way of limitation, polysaccharides, polyesters, polyamino acids, homopolymers, polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release.

Another possible method useful in controlling the duration of action of the active agents by controlled release preparations and half-life is incorporation of the active agents or their functional derivatives into particles of a polymeric material such as polyesters, polyamides, polyamino acids, hydrogels, poly(lactic acid), ethylene vinylacetate copolymers, copolymer micelles of, for example, polyethylene glycol (PEG) and poly(l-aspartamide).

Additional pharmaceutical methods may be employed to increase bioavailability of the drug, such as Kolliphor HS15.

It is also possible to entrap the active agents in microcapsules prepared, for example, by coacervation techniques or by interfacial polymerization (for example, hydroxymethylcellulose or gelatine-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are well known to persons having ordinary skill in the art.

When the active agents are to be used as an injectable material, they can be formulated into a conventional injectable carrier. Suitable carriers include biocompatible and pharmaceutically acceptable phosphate buffered saline solutions, which are particularly isotonic.

For reconstitution of a lyophilized product in accordance with the present disclosure, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above for the reconstitution of a lyophilized product.

The active agents can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, tauric acid, maleic acid, and fumaric acid, or by reaction with an inorganic base such as sodium hydroxide, ammonium hydroxide, potassium hydroxide, and organic bases such as monoalkyl, dialkyl, trialkyl and aryl amines, and substituted ethanolamines.

In certain non-limiting embodiments, the present disclosure includes an active agent composition wherein at least one of the active agents is coupled (e.g., by covalent bond) directly or indirectly to a carrier molecule.

Formulated compositions comprising the active agents of the present disclosure can be provided in unit dosage form, e.g., in ampoules or in multi-dose containers, with an added preservative. Compositions can also take such forms as suspensions, solutions, or emulsions in oily or aqueous vehicles, and can contain formulatory agents such as suspending, stabilizing, and/or dispersing agents.

In some non-limiting methods, the patient is administered the active agent every one, two, three, or four weeks, for example. The dosage depends on the frequency of administration, condition of the patient, response to prior treatment (if any), whether the treatment is prophylactic or therapeutic, and whether the disorder is acute or chronic, among other factors.

The number of dosages administered may depends on the severity and temporal nature of the disorder (e.g., whether presenting acute or chronic symptoms) and the response of the disorder to the treatment. For acute disorders or acute exacerbations of a chronic disorder, between 1 and 10 doses may be used. Sometimes a single bolus dose, optionally in divided form, is sufficient for an acute disorder or acute exacerbation of a chronic disorder. Treatment can be repeated for recurrence of an acute disorder or acute exacerbation. For chronic disorders, the active agent may be administered at regular intervals, such as (but not limited to) weekly, fortnightly, monthly, quarterly, every six months for at least 1, 5, or 10 years, or for the life of the patient.

Compositions can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day. The duration of treatment (i.e., the period of time over which the composition is administered) can vary, depending on any of a variety of factors, e.g., subject response. For example (but not by way of limitation), the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The dosage of an administered active agent for humans will vary depending upon factors such as (but not limited to) the patient's age, weight, height, sex, general medical condition, and previous medical history. A dosage may be provided as several smaller amounts. For example, a single dosage of 500 mg may be administered as ten 50 mg tablets or capsules, or as five 100 mg tablets or capsules. The amounts of doses or dosages described herein may be provided in a single capsule, tablet, injection, infusion, or other more of delivery. Or, the amounts of drug which comprise the doses or dosages described herein may be provided in two or more (e.g., 2, 3, 4, 5, 6, 7, 8, 9, 10, 11, or 12) capsules, tablets, injections, infusions, or other modes of delivery.

In some non-limiting embodiments, a per capsule or tablet dose may range from about 25 mg to about 200 mg (e.g., from about 25 mg to about 175 mg, from about 25 mg to about 150 mg, from about 25 mg to about 125 mg, from about 25 mg to about 100 mg, from about 25 mg to about 75 mg, from about 25 mg to about 70 mg, from about 40 mg to about 100 mg, from about 40 mg to about 75 mg, from about 40 mg to about 175 mg, from about 40 mg to about 150 mg, from about 40 mg to about 125 mg, from about 40 mg tov 70 mg, from about 40 mg to about 60 mg, or from about 45 mg to about 55 mg). In some non-limiting embodiments, the dose per capsule or tablet may be about 50 mg.

In some non-limiting embodiments, the dose per capsule or tablet is from about 20 mg to about 200 mg (e.g., from about 20 mg to about 175 mg, from about 20 mg to about 150 mg, from about 20 mg to about 100 mg, from about 20 mg to about 75 mg, from about 30 mg to about 175 mg, from about 40 mg to about 175 mg, from about 45 mg to about 175 mg, from about 50 mg to about 175 mg, from about 30 mg to about 150 mg, from about 40 mg to about 100 mg, from about 45 mg to about 75 mg, from about 50 mg to about 70 mg, from about 55 mg to about 65 mg, from about 58 mg to about 62 mg; about 20 mg, about 30 mg, about 45 mg, or e.g., about 60 mg). In some non-limiting embodiments, the dose is from about 12 mg to about 48 mg (e.g., from about 12 mg to about 42 mg, from about 12 mg to about 36 mg, from about 12 mg to about 30 mg, from about 18 mg to about 48 mg, from about 18 mg to about 42 mg, from about 24 mg to about 42 mg, from about 27 mg to about 42 mg, from about 24 mg to about 36 mg, from about 27 mg to about 33 mg, from about 28 mg to about 32 mg; e.g., about 24 mg, about 27 mg, about 30 mg, about 33 mg, or about 36 mg).

In some non-limiting embodiments, the dosing regimen comprises administration of a loading dose, such as from about 20 mg to about 200 mg (e.g., from about 20 mg to about 175 mg, from about 20 mg to about 150 mg, from about 20 mg to about 100 mg, from about 20 mg to about 75 mg, from about 30 mg to about 175 mg, from about 40 mg to about 175 mg, from about 45 mg to about 175 mg, from about 50 mg to about 175 mg, from about 30 mg to about 150 mg, from about 40 mg to about 100 mg, from about 45 mg to about 75 mg, from about 50 mg to about 70 mg, from about 55 mg to about 65 mg, from about 58 mg to about 62 mg; e.g., about 60 mg). In some non-limiting embodiments, the dose is from about 12 mg to about 48 mg (e.g., from about 12 mg to about 42 mg, from about 12 mg to about 36 mg, from about 12 mg to about 30 mg, from about 18 mg to about 48 mg, from about 18 mg to about 42 mg, from about 24 mg to about 42 mg, from about 27 mg to about 42 mg, from about 24 mg to about 36 mg, from about 27 mg to about 33 mg, from about 28 mg to about 32 mg; e.g., about 24 mg, about 27 mg, about 30 mg, about 33 mg, or about 36 mg).

In certain non-limiting embodiments, the per day dosage for administration to a subject is in a range of about 1 mg/kg to about 25 mg/kg, or about 2 mg/kg to about 24 mg/kg, or about 3 mg/kg to about 22 mg/kg, or about 4 mg/kg to about 20 mg/kg, or about 5 mg/kg to about 17.5 mg/kg, or about 5 mg/kg to about 15 mg/kg, or about 5 mg/kg to about 12.5 mg/kg, or about 6 mg/kg to about 25 mg/kg, or about 6 mg/kg to about 20 mg/kg, or about 6 mg/kg to about 15 mg/kg, or about 6 mg/kg to about 12 mg/kg.

In certain non-limiting embodiments, the amount of the active agent delivered to the subject per dose is in a range of about 100 mg to about 1000 mg, or about 200 mg to about 1000 mg, or about 300 mg to about 1000 mg, or about 400 mg to about 1000 mg, or about 250 mg to about 900 mg, or about 400 mg to about 900 mg, or about 400 mg to about 850 mg, or about 400 mg to about 800 mg, or about 400 mg to about 700 mg, or about 420 mg to about 840 mg, or about 500 mg to about 650 mg, or about 500 mg to about 1000 mg, or about 500 mg to about 900 mg, or about 500 mg to about 850 mg, or about 500 mg to about 800 mg, or about 500 mg to about 700 mg. The total dose to be delivered can be provided in a single capsule, tablet, injection, or bolus (or other dosage form), or in multiple capsules, tablets, injections, or boluses (or other dosage forms).

In at least certain non-limiting embodiments, when the active agent is provided in the form of a capsule or tablet, the capsule or tablet should disintegrate within about 5-10 minutes, and in the gastrointestinal (GI) tract the active agent should dissolve in about 30 minutes.

While several embodiments have been provided in the present disclosure, it may be understood that the disclosed methods might be embodied in many other specific forms without departing from the spirit or scope of the present disclosure. The present description is to be considered as illustrative and not restrictive, and the intention is not to be limited to the details given herein. In addition, techniques and methods described and illustrated in the various embodiments as discrete or separate may be combined or integrated with other techniques or methods without departing from the scope of the present disclosure. Various substitutions and alterations are ascertainable by one skilled in the art and may be made without departing from the spirit and scope disclosed herein.

What is claimed is:

1. A method of treating or mitigating traumatic brain injury in a subject in need of such treatment, comprising:
administering to the subject at least one Nociceptin/Orphanin FQ (N/OFQ) peptide receptor (NOP receptor) partial agonist or antagonist.

2. The method of claim 1, wherein the at least one NOP receptor partial agonist is selected from the group consisting of PPL-138 (BU10038), AT-035, AT-001, AT-104, SER100 (ZP120), and combinations thereof.

3. The method of claim 1, wherein the at least one NOP receptor partial agonist is PPL-138 (BU10038).

4. The method of claim 1, wherein the at least one NOP receptor partial agonist is AT-035.

5. The method of claim 1, wherein the at least one NOP receptor partial agonist is AT-001.

6. The method of claim 1, wherein the at least one NOP receptor partial agonist is AT-104.

7. The method of claim 1, wherein the at least one NOP receptor partial agonist is SER100 (ZP120).

8. The method of claim 1, wherein the at least one NOP receptor antagonist is selected from the group consisting of LY2940094, MK-5757, JTC-801, SB612111, and combinations thereof.

9. The method of claim 1, wherein the at least one NOP receptor antagonist is LY2940094.

10. The method of claim 1, wherein the at least one NOP receptor antagonist is MK-5757.

11. The method of claim 1, wherein the at least one NOP receptor antagonist is JTC-801.

12. The method of claim 1, wherein the at least one NOP receptor antagonist is SB612111.

13. The method of claim 1, wherein the subject is also in need of treatment for post-traumatic stress disorder (PTSD).

14. A method of treating or mitigating traumatic brain injury (TBI) and post-traumatic stress disorder (PTSD) in a subject in need of such treatment, comprising:
administering to the subject at least one Nociceptin/Orphanin FQ (N/OFQ) peptide receptor (NOP receptor) partial agonist or antagonist.

15. The method of claim 14, wherein the at least one NOP receptor partial agonist is selected from the group consisting of PPL-138 (BU10038), AT-035, AT-001, AT-104, SER100 (ZP120), and combinations thereof.

16. The method of claim 14, wherein the at least one NOP receptor antagonist is selected from the group consisting of LY2940094, MK-5757, JTC-801, SB612111, and combinations thereof.
